(19) United States
(12) Patent Application Publication
(10) Pub. No.: US 2010/0059063 A1
(43) Pub. Date: Mar. 11, 2010

(54) Long acting germicidal condom and manufacture procedure

(76) Inventor: Chih-Yao Wu, Taipei (TW)

Correspondence Address: Wu, Chih-Yao, P.O.Box 166-13, Taipei, 115, TW

(21) Appl. No.: 12/230,891

(22) Filed: Sep. 8, 2008

(51) Int. Cl. A61F 6/04

(52) U.S. Cl. 128/844

(57) ABSTRACT

The present invention is to provide a long-acting germicidal condom and manufacture procedure. A long acting germicidal condom comprises a sheath (1) has a filmy structure to protect male sexual organ, said sheath (1) includes an extruded latex (12) combined with a nano scale titanium oxide photocatalysis coating liquid (2), a nano scale zinc oxide (3) and a nano scale chitin beads and chitosan beads (4); a lubricating germicidal solution (5) spread on a cylindrical surface about said sheath (1); and an O ring (6) coupled to an open end of said sheath (1).

### FIELD OF THE INVENTION

The present invention is related to a long-acting germicidal condom and manufacture procedure.

### DESCRIPTION OF PRIOR ART

Changes of the social morality and environment, today we are facing more sexual transmitted diseases (STDs) than ever, such as acquired immunodeficiency syndrome (AIDS), syphilis and gonorrhea; among all STDs, AIDS is the most terrified epidemic. On Dec. 1, 2006, the 19^{th} World AIDS Day, a report of the Voice of America (VOA) in Hong Kong, World Health Organization (WHO) warned all Asia countries that All Asia governments must take actions immediately to stop the AIDS, otherwise the AIDS will become serious problem in Asia. Today, more than 8 million people in Asia are infected with human immunodeficiency virus (HIV), and patient population estimate is still rising.

Also, WHO had warned that unsafe sexual intercourse has helped the AIDS spread rapidly in Asia. According the official report of China, from January to October of this year, people infected with HIV are 30% higher in comparison with the same duration of last year.

According the specialist, this data reflects that most people in Asia don't have necessary and sufficient information about the AIDS, because sex still is a taboo in most eastern societies, it is useless speaking, and receives much less attention; most governments chose unintentionally ignore and not to face the problem squarely.

Laval University in Canada discovered that sodium dodecyl sulfate (SDS) or Lauryl sodium sulfate (SLS) (i.e. C₁₂H₂₅SO₄Na) contained in household toothpaste may form an “invisible condom” for woman, since it may be a liquefied paste outside the human body, but it became a gelatinous material to separate infectious HIV from filtrating into the victim's body tissue. And, animal tests showed positive effects on such hypothesis.

“With a vaccine not immediately on the horizon, microbicides that can remove cholesterol from cell membranes, rendering HIV non-infectious, may play an important part in controlling the AIDS pandemic”, researchers of John Hopkins school of medicine discovered new way to block HIV transmission.

In 2006, Gates foundation donated 500 million dollars to The Global Fund to fight AIDS, Tuberculosis, and Malaria (GFATM), Bill Gates said that the “best hope” for limiting the spread of HIV/AIDS over the next 10 years would be the development of a microbicide. Microbicides include a range of products—such as gels, films, sponges and other products—that could help prevent the sexual transmission of HIV and other STDs.

“Arrival on the market of generic versions of anti-HIV/AIDS drugs was what enabled the cost of tritherapies to be reduced from USD 10 000 per patient per year in 2000 to the current USD 130 per patient per year” cheap but effective measures may be adopted in such a manner in February 2007 understood by EP parliament. It is expected that 95% survived HIV infected populace in Asia, for example, up to 20% of China's infected patients may have gotten free medicine, now could be a more sustainable population development.

Under such circumstances, at least, some “invisible condom” made from microbiocide gel developed during the past decade might have passed research test on human body to be accepted into market in 2009, if possible. The 16^{th} international AIDS conference, Toronto 2006, made us wait for those integrated developments for preventing the AIDS epidemic.

In fact, prevention of STDs, conventional condom is still the most effective method to prevent STDs. Using condom during the complete sexual intercourse can provide best protection.

Such as U.S. Pat. No. 6,892,732 entitled “Condom coated with lactic acid bacteria and contained composition” to Wang, Chih-Hong et al., on May 17, 2005 (hereinafter '732), it disclosed a condom coated, for example, micro-encapsulated form lyophilized lactic acid bacteria (LAB), by plating the quantified dilution of bacteria on to blood agar plates or other enriched media, incubating at 37 degree C., for 24-48 hours in a 5-10% carbon dioxide atmosphere, and then performing a colony count. The removal of the nutrient media is done by centrifugation at 14,000 times at 0-4 degree C., and then washing with sterile, balanced salts and 5% glucose solution at least three times after the initial centrifugation. It contains between one to ten billion organisms per ml, at 0-4 degree C., Being forced through needle under pressure, droplets are collected in an aqueous solution of 1.3-2% calcium chloride where they gel, further washed with at least a five fold excess of the 0.1% CHES 1.1% calcium chloride, and normal saline solution. The resultant spheres are then “snap frozen” in liquid nitrogen and then lyophilized.

LAB are usually delivered as living, potentially beneficial, bacterial cells to the eater's gut as non-digestible carbohydrates delivered in food to the large bowel to provide fermentable substrates for selected bacteria, however, when such bacteria provided to the condom—although the same may be designed as edible—but how, was not clearly taught in '732.

Or as published US 20070000020 entitled “Surgical glove with modified frictional interface and method for producing same” to Maris Vistins et al., on Jan. 4, 2007 (hereafter '020). It disclosed that surgeon's gloves, should not restrict the natural movement and flex of a wearer's hands and wrist. Further, glove-cuff slippage does not constrict the glove cuff, nor substantially change the gross physical configuration of the glove from original designs. To increase mechanical or frictional engagement of one surface with another surface or friction when worn, a colloidal particle-containing slurry or an abrasive-impregnated substrate to an exposed elastomeric matrix surface to create a rough, textured or scabrous surface with a topography of micron-scale ridges and dimples is to provide slip resistance without forming a permanent bond between the elastomeric surface and bare skin normally. An abrasive substrate may be a roughened sheet, fabric strip, or set of bristles, impregnated or studded with a layer of abrasives composed of, for example, a) silicates, such as silica (SiO₂) or garnet, b) aluminum oxides (Al₂O₃), such as corundum or fused alumina, and/or c) silicon carbide.

In '020, various shaped fumed soot particles, or fused silica particles are applied to the textured surface, the overall particle size distribution is in the range about 10-600 nm, but the variation in particle size of individual particles is demanded clustered closely round a mean value. Particle-size distribution adjusted to control the final surface finish, where residue abrasive particles processing a wash or rinse, substantially an acid wash, to remove any residual colloidal particles is required. If all processing are not completed prior to the abrasion, the post treatments will adversely affect the abraded area. Thus, labor cost and material cost devoted in '020 may supply exceed demand, and, especially, acid wash is not recognized environmental friendly.

Accordingly, the present invention is to provide a condom to inhibit bacteria growth and manufacturing the same targeting human needs.

### SUMMARY OF THE INVENTION

Method of manufacturing a long-acting germicidal condom comprises the steps of:

First step: a nano scale titanium dioxide photocatalysis coating liquid (2) to emit far infrared rays, a nano scale zinc oxide (3) with germicidal effect, and a nano scale chitin beads and chitosan beads (4) are combined to form a composite material (10);

Second step: said composite material (10) is mixed into an elastomer material (11) to form a latex (12);

Third step: said latex (12) with a density is extruded to form a sheath (1) further integrally coupled with an O ring;

Fourth step: a cylindrical surface of said sheath (1) is spread with lubricating germicidal solution (5); thus to finish a long acting germicidal condom.

Method of manufacturing a long-acting germicidal condom of claim 1 wherein said nano scale titanium dioxide photocatalysis coating liquid (2) emits said far infrared rays wave length in the range of 6-12 μm.

Method of manufacturing a long-acting germicidal condom of claim 1 wherein said lubricating germicidal solution (5) is a mixture of said nano scale zinc oxide (3) and said nano scale chitin beads and chitosan beads (4).

Method of manufacturing a long-acting germicidal condom of claim 1 wherein said elastomer material (11) selected from one of the following: natural rubber, synthetic rubber.

A long acting germicidal condom comprises a sheath (1) has a filmy structure to protect male sexual organ, said sheath (1) includes an extruded latex (12) combined with a nano scale titanium oxide photocatalysis coating liquid (2), a nano scale zinc oxide (3) and a nano scale chitin beads and chitosan beads (4);

A lubricating germicidal solution (5) spread on a cylindrical surface about said sheath (1); and

An O ring (6) coupled to an open end of said sheath (1).

The long acting germicidal condom of claim 5 wherein said nano scale titanium oxide photocatalysis coating liquid (2) emits far infrared rays wave length in the range of 6-12 μm.

The long acting germicidal condom of claim 5 wherein said lubricating germicidal solution (5) is a mixture of said nano scale zinc oxide (3) and said nano scale chitin beads and chitosan beads (4).

The long acting germicidal condom of claim 5 wherein said elastomer material is selected from one of the following: natural rubber, synthetic rubber.

### ADVANTAGES OF THE EMBODIMENTS OF THE INVENTION

Advantages and benefits can be attained by embodiments of the present invention as following:

1. Said nano scale titanium dioxide (2) photocatalysis coating liquid mixed into said sheath (1). As well known, said titanium dioxide (2) can resist mould or mildew on wall effectively. Once said titanium dioxide (2) is spread over on the wall, mildew of fungus will not grow, however, other than the titanium dioxide (2) painted area, wherever the wall will be doomed to grow with mildew or fungus later, when filtrated by rain water or the like. Ultra violet light may trigger said titanium dioxide (2) to dissolve organic materials and bacteria. Further, said emission of far infrared rays may improve blood circulation in human body, jerks or sport injuries will not be caused easily to users. Such a titanium dioxide (2) lowers the possibility of infection, and may be a more comfortable change for promotion of safety sex.

2. In addition, said nano scale zinc oxide (3) has high catalytic efficiency and strong adsorption ability; further its high isoelectric point (9.5), biocompatibility, and fast electron transfer kinetics, the use of zinc oxide as a biomimic membrane immobilize and modify biomolecules, also a necessary nutrient can be added to the food.

3. Chitin material wears away with time as the wound heals so that it can be used as surgical threads to accelerate healing of wounds. Chitin used as a stand-alone wound-healing agent. Therefore, nano scale chitin beads and chitosan beads (4) added to said sheath (1) also benefit the couples likely a preemptive threapy.

4. Said lubricating germicidal solution (5) is applied to the cylindrical surface of the sheath (1), that is not only an invisible condom like SDS or SLS namely a placebo extracted from toothpaste, but also the real lubricating germicidal solution added with germicidal effect makes the condom beneficial for both male and female also lower the infection rate to a minimum level.

5. Whenever the sheath (1) is exposed outside its package, 24 hours long-acting effectiveness is expected to sustain safety for both partners to whom the conventional or referenced condom can't compare.

### BRIEF DESCRIPTION OF DRAWINGS

FIG. 1 is a perspective view of the condom of the present invention.

FIG. 2 is a cross sectional view of the condom of the present invention.

FIG. 3 is an enlarged view of FIG. 2 indicated by a circle.

FIG. 4 is a diagrammatic view showing block diagram of the manufacturing procedure.

FIG. 5 is a diagrammatic view showing block diagram of the manufacturing process.

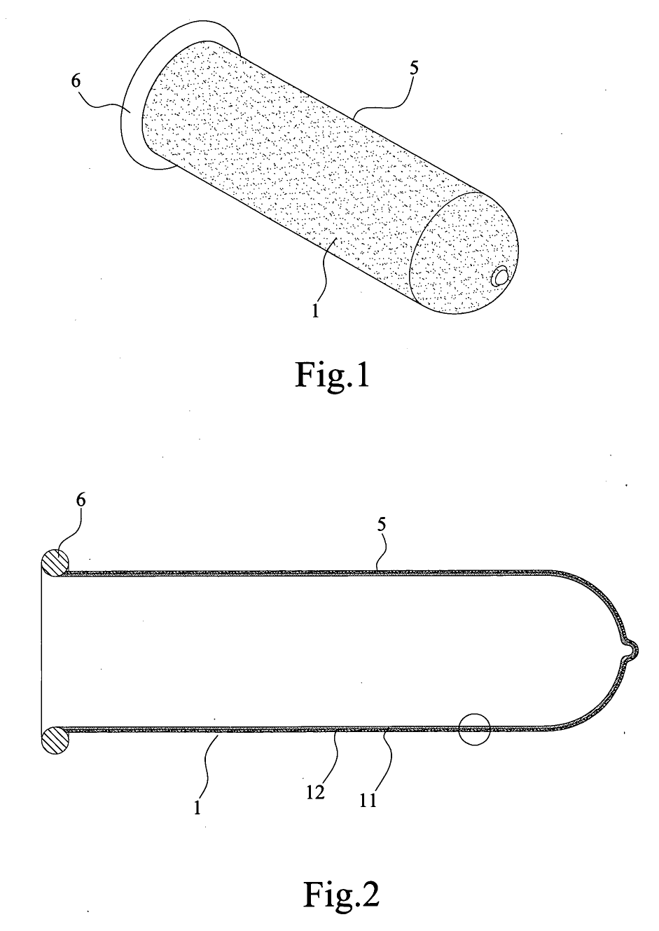

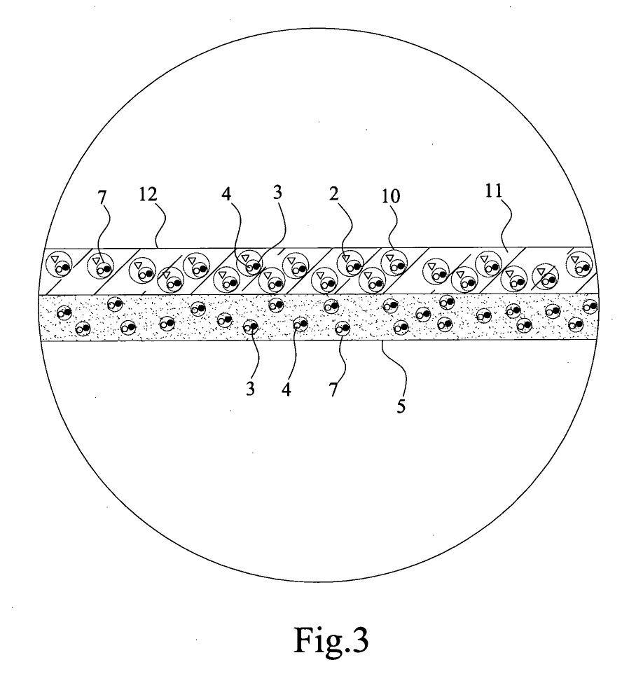

### DETAIL DESCRIPTION OF THE PREFERRED EMBODIMENT OF THE PRESENT INVENTION

The description is described in detail according to the appended drawings hereinafter.

As shown in FIG. 13, a long-acting germicidal condom comprises a sheath (1) is a tubular filmy structure that fits over reproductive organ of a man to protect private couple from acquired immunodeficiency syndrome (AIDS) infection or the like, even for fertile married couple having regular unprotected intercourse. The sheath (1) made of a latex (12) is extruded out by an extrusion press. Said latex (12) is composed of an elastomer material (11) mixed with a nano scale titanium dioxide (2) photocatalysis coating liquid, nano scale zinc oxide (3) and nano scale chitin beads and chitosan beads (4).

A lubricating germicidal solution (5) is applied to along a cylindrical surface of the tubular sheath (1); and an O-ring which is coupled to an open end of the tubular sheath (1).

The present invention is to emphasize the condom with said nano scale titanium dioxide (2) photocatalysis coating liquid. Titanium is a chemical element with the symbol Ti and atomic number 22. It is a light, strong, lustrous, corrosion-resistant transition metal with a metallic white color. Titanium based alloy exhibits “super” properties, such as ultrahigh strength and super elasticity. The new materials could prove useful for spaceflight, where precision operations are conducted in ruthless conditions. People dubbed titanium alloy as “space age metal”.

Said nano scale titanium dioxide (2) photocatalysis coating liquid can be attributed to alkaline ion water. Our blood pH must be maintained in a very narrow range (pH=7.3-7.45). In order to maintain our body blood alkalinity, give the body alkaline ionized water instead of readily available source of the body-loss of phosphorous and calcium may cause “osteoporosis”, reducing magnesium and potassium may cause leg cramps. Further, said nano scale titanium dioxide emits wavelength of 6-12 μm far infrared rays can increase blood circulating rate. In addition, our cells build a wall of fat around them to fend off the acid, when we alkalize the body to remove all that acid and the cells lower their fat content. Cells may be activated in the weightless compartment to exchange oxygen and carbon dioxide.

Mix the nano scale titanium dioxide (2) photocatalysis coating liquid as far infrared ingredients, the radiation weave from the far infrared ingredients is between 6˜12 μm and it is close to the 9.36 μm, which is released from human beings. The radiation thus can go deep into hypoderm and resonate water molecules in our body then dilate the vein by vibrating water molecules. The water molecules on the surface of cell are active so it will accelerate the flow of calcium ion then advance blood circulation, improve metabolism, active cell tissue, accelerate absorption of oxygen.

In addition, said nano scale zinc oxide (3) has high catalytic efficiency and strong adsorption ability; further its high isoelectric point (9.5), biocompatibility, and fast electron transfer kinetics, the use of zinc oxide as a biomimic membrane immobilize and modify biomolecules, also a necessary nutrient can be added to the food.

Chitin material wears away with time as the wound heals so that it can be used as surgical threads to accelerate healing of wounds. Chitin used as a stand-alone wound-healing agent. Therefore, nano scale chitin beads and chitosan beads added to said sheath (1) also benefit the couples likely a preemptive threapy.

A layer of antibiotic and lubricating germicidal solution (5) is applied to the cylindrical surface of the sheath (1), that is not only an invisible condom like SDS or SLS namely a placebo extracted from toothpaste applied thereto, but also the real lubricating germicidal solution added with germicidal effect makes the condom beneficial for both male and female also lower the infection rate to a minimum level.

Said elastomer material (11) is selected from one of the following: natural rubber, synthetic rubber.

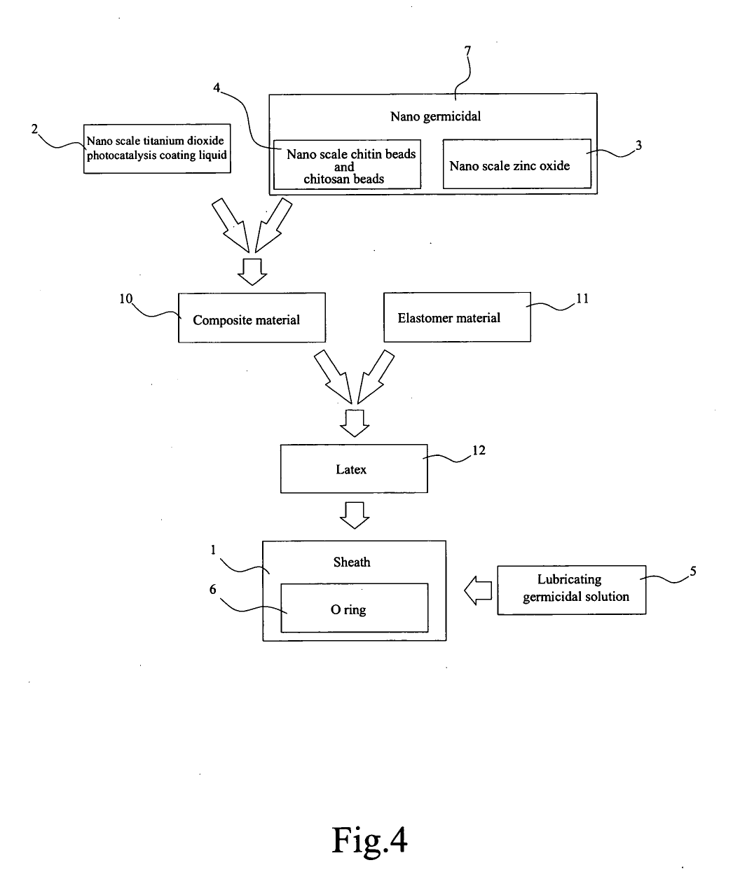

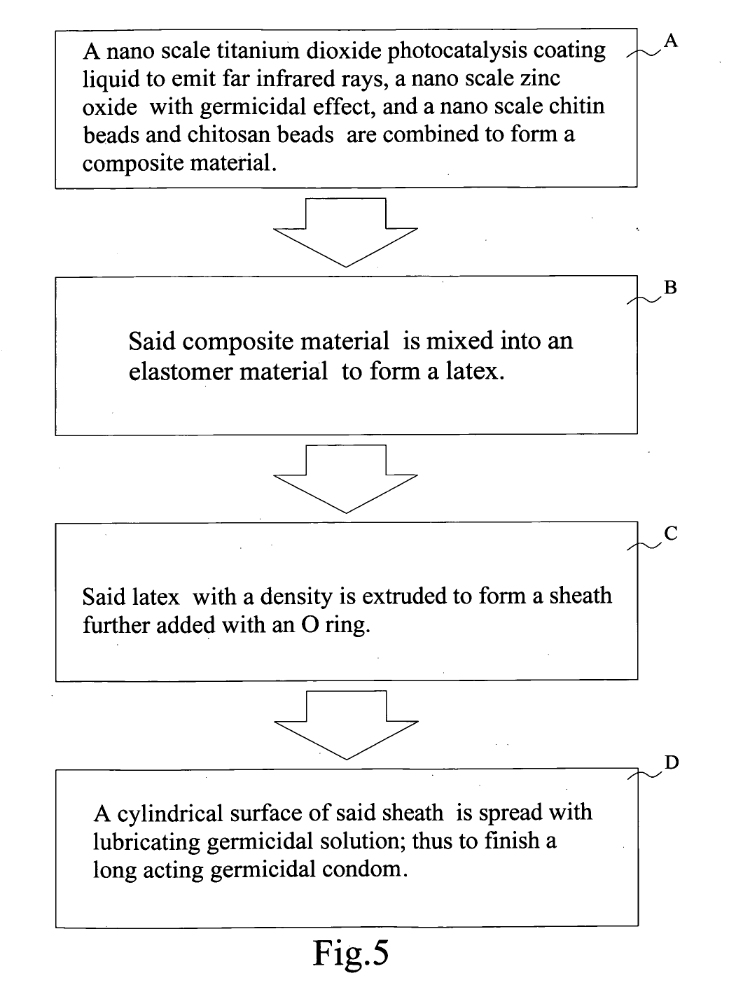

As shown in FIGS. 4-5, diagrammatic views showing block diagrams of the manufacturing procedure are illustrated.

Method of manufacturing a long-acting germicidal condom comprises the steps of:

First step: said nano scale titanium dioxide photocatalysis coating liquid (2) to emit far infrared rays, said nano scale zinc oxide (3) with germicidal effect, and said nano scale chitin beads as well as said chitosan beads (4) are combined to form said composite material (10);

Second step: said composite material (10) is mixed into said elastomer material (11) to form said latex (12);

Third step: said latex (12) with a density is extruded to form said sheath (1) further added with said O ring;

Fourth step: said cylindrical surface of said sheath (1) is spread with said lubricating germicidal solution (5); thus to finish said long acting germicidal condom.

Nano scale zinc oxide (3) is endowed with germicidal effect as the size of the system decreases. These include statistical mechanical effects, as well as quantum mechanical effects, for example the “quantum size effect” where the electronic properties of solids are altered with great reductions in particle size. Especially, under ultra violet irradiation, zino oxide can release electron (e⁻) and hole (h⁺) pair from air and water. Said hole (h⁺) may convert oxygen in the air into activated oxygen, which is further reacted with various organic materials. Most bacteria and viruses killed. High efficiency of light absorption of the nano scale zince oxide (3) results in germicidal effect.

Chitin beads and chitosan beads is polysaccharide known to be natural polymers, non-toxic, biodegradable and biocompatible. Chitosan is hypoallergenic, and has natural anti-bacterial properties. Including growth of staphylococcus aureus, candida albicans, fongus, and bacillus aerginosa can be inhibited.

Nano scale chitin beads partly deacetylated, contains cationic group on the polyglucosamine group may interfere with anionic group on surfaces of the bacteria. Amino group of the chitosan or deacetylated chitin diluted in acid may convert into positive charged ammonia (NH3), which affects liquid permeability to cell wall of the bacteria, results in cytoplasma inside the cell seeping out.

Since said sheath (1) imbued with said nano scale zinc oxide (3), said nano scale chitin beads and chitosan beads (4), and said nano scale zinc oxide (3) as well as said nano scale chitin beads and chitosan beads contained in said lubricating germicidal solution (5), all of the nano scale beads and oxides are collectively called nano germicidal (7). Therefore, targeting to inhibit growth of the bacteria, said nano germicidal (7) is to be directly coupled to cell wall of said bacteria, and invade into the bacteria to inhibit both metabolism and activity of enzyme.

In other words, said nano scale zinc oxide (3) mixed with said nano scale chitin beads and said chitosan beads (4) to form a mixture as said nano germicidal (7), distinct attribute of which listed as following:

Ingredients are examined by super laboratory supervised by Yang-Ming University incubation center since 2001 and approved by accompanied, if any, identification number experiment report:

ionicity: non-ionicity

pH value: 8.5±1

appearance: transparent

function: germicidal, inhibition of mould growth

category: nano scale inorganic germicidal

means: detergent contact

effect: 100% bacteria extermination at a time through the duration up to one hour

temperature range: broadly applied to couple beyond the range of normal room temperature and body temperature

effective duration: static detergent contact, long-acting

exposure time of germicidal: 2 minutes-24 hours, adjusted as demanded

non-toxicity: test of eight heavy metal included lead, barium, cadmium, chromium, antimony, mercury, arsenic, selenium contained lower than 0.25-1.5 ppm (approved by Tai-Am SL 94E9080)

take orally: passed the examination under Taiwan accreditation foundation (TAF) recognized international standard (approved by Tai-Am SL 94E90117)

test on bare human skin: passed the examination under TAF recognized international standard (approved by Tai-Am SL 94E90117)

containing formaldehyde test: none (approved by Tai-Am SL 94E9080)

on shelf availability: 5 years (approved by Tai-Am SL 95E90020)

heat stability: endurable processed through 600° C. higher temperature

Moreover, germicidal is effective against bacteria listed as following: Bacillus subtilis, Tubercle bacillus, Mycobacterium fortuitum, Candida albicans, Straphylococcus aureus, Aspergillus niger var., Legionella pneumophila, Methicillin (meticillin) resistant straphylococcus aureus (MRSA), Drug resistance strain of bacillus aerginosa, Influenza hemophilus, Streptococcus pneumoniae, Neisseria meningitides, Acinetobacter baumannii, Trichopyton mentagrophytes, Propionibacterium acnes, Penicillium var, Chaetomium globosum, Aureobasidiumpululans, Gliocladium virens

Based on said super laboratory's report, said nano scale zinc oxide (3) mixed with said nano scale chitin beads and nano scale chitosan beads (4) to form a mixture of said nano germicidal (7) is effective to inhibit growth of over ten bacteria strains. Test on bare human skin further approved by said super laboratory. Non-toxic nano germicidal (7) is endowed with a certain oxidation-reduction capacity so as to inhibit growth of bacteria.

Said nano germicidal (7) of low-density (for example, diluted to over one fortieth density) still can be effectively coupled to enzymes of prolease of cell membrane to inhibit activity of said enzymes and metabolism of said cell.

Uses of most medicine with diminished capacities, when coupled to said bacteria, soon will be dissipated by physical process, such as dissolved into water or the like and carried away with perspiration, urine, or the like. However, said nano germicidal (7) may still filtrate cells to inhibit growth of said bacteria.

Treatments for infection of said mould of multi cell fungi, said nano germicidal (7) remained a composite prescription of said mixture of said nano scale zinc oxide (3) and said nano scale chitin beads and said nano scale chitosan beads (4). The use of nano germicidal (7) is designed as a controlled release dosage form.

Slow release or timed release of said nano germicidal (7) is designed effective up to five years on shelf availability. Diameters of said nano zinc oxide (3) or said nano scale chitin beads and said nano scale chitosan beads (4) may determine whether they are retained inside said nano germicidal (7) for a longer while in use, also germicidal efficacy of said zinc oxide (3) and said chitin as well as said chitosan (4) may be individually affected is under consideration.

Said controlled release dosage form is significant as timed release at a preordained speed and effective through a certain regulated period. Thus said nano germicidal (7) may be kept effective a longer while within a selected range of diluted density.

Generally, said controlled release dosage may be effective with the half time in the range of 2-12 hours, but most of conventional germicidal is given with immediate germicidal effect.

With a short germicidal duration, for example, alcohol used as a germicidal must be applied to the same human body many times. Obviously, all germicidal materials necessitate “long acting” mechanism. Or germicidal materials of less bioavailability are not suitable for generating “long acting” dosages.

A significant germicidal effect is preferably long acting, extensive and effective exposure, to come out first, no other choices considered.

On the market, most germicidal ingredients may cause drug resistance strains arise. Diagnoses and cures on bacterial infectious diseases are more lengthy and tedious on the potential risk caused by the various strains. To create germicidal of new age was at issue.

The use of said nano germicidal (7) replaces conventional germicidal prescription can be a distinctive attribute compared to, for example, alcohol-based hand gel or the like.

Outside cell membrane of said fungi is a layer of solid cell wall plotted against effective filtration pressure from inside of said bacteria. Thus, normal cell type can be retained.

Cell wall is mainly composed of mucopeptide, N-acetylglucosamine (GNAC) and N-acetylmuranic acid (MNAC) cross linked by peptide chain.

Biosynthesis of said mucopeptide can be decomposed into three stages: synthesis of prosoma (precursor or stem cell of a plasma cell) inside cytoplasma stage, N-acetylmuramyl pentapeptide and acetylgloucosamine links created on plasma membrane stage, and translocating peptides outside cytoplasma stage to form cross links. Said nano germicidal (7) can inhibit synthesis of said mucopeptide during inside cytoplasma stage.

Timed release of said nano germicidal (7) may inhibit synthesis of said N-acetylmuranic acid due to released ingredients inhibit ligase and racemase of D-alanine. Synthesis of said mucopeptide during plasma membrane stage can be destroyed by said nano germicidal (7) in combination.

With said nano germicidal (7), synthesis of linear decapeptide disaccharide polymer formed by N-acetylmuramyl pentapeptide coupled to carrier of fat, process of said polymer translocation to receptors outside of said plasma membrane, and a dephosphoric reaction are respectively inhibited.

Said nano germicidal (7) further can inhibit cross links of said linear decapeptide disaccharide polymer outside of cytoplasma.

Target sites of said nano germicidal (7) are binding proteins (BPs) on plasma membrane results in translocating peptides of transferase to inhibit cross links.

Said nano germicidal (7) can inhibit synthesis of plasma cell, and cause defects on cell wall.

Since high hydrostatic filtration pressure inside said bacteria that usually have equal osmotic pressure under isotonic environment, water filtrates into said bacteria, which are therefore expanded and deformed, further affected in autolysis enzymes disorder, said bacteria broken open to be dissolved into water.

Plasma membrane of said fungi, a semi-permeable membrane, consists of lipoid and protein molecules, acts as permeability barrier and material relocation.

Said nano germicidal (7) has surfactant materials to be selectively coupled to phospholipids (PL) of the plasma membrane.

Said nano germicidal (7) has nano-photocatalyst polyene to be coupled to sterol materials of the plasma membrane of fingi.

Permeability of said plasma membrane is therefore increased, that leads to proteins, nucleotides, amino acids, saccharides, and salts to be leaked out to kill said bacteria.

Bacteria are prokaryotes, ribsomes of which is 70S, composed of 30S and 50S subunits. Mammals have eukaryotes, ribsomes of which is 80S, composed of 40S and 60S subunits. Significant difference was found between ribsomes of both eukaryotes and prokaryotes on physiological, biochemical, and functional aspects.

Ingredients of said nano germicidal (7) are given with highly selected toxicity to ribsomes of bacteria, but not do harm to synthesis of ribsomes and proteins of mammals.

Nano germicidal (7) can inhibit synthesis of protein of bacteria in the different ways as following:

1. Said nano germicidal (7) coupled to 50S subunit of ribsomes of said bacteria, proceeds “reversal inhibition” on synthesis of protein of bacteria.

2. Said nano germicidal (7) coupled to 30S subunit of ribsomes of said bacteria, said nano germicidal (7) for inhibition of growth of bacteria can inhibit aminoacyl coupled to A site of 30S subunit of the ribsome to inhibit synthesis of protein.

3. Said nano germicidal (7) coupled to said 30S subunit of the ribsome is multi-linked combination to affect all process of synthesis of protein to inhibit growth of bacteria.

Composite prescription of said nano germicidal (7) can inhibit synthesis of deoxyribonucleic acid (DNA), at the same time, ribonucleic acid (RNA) polymerase requires DNA template is inhibited.

Besides, said nano germicidal (7) is adopted to replace conventional titanium dioxide, a photocatalyst limited by light source environment, whenever there is darkness, no germicidal effect can be expected. But, said nano germicidal (7) exposed to darkness still can show deodorization and germicidal effect up to 24 hours.

Sufficient said nano germicidal (7) contained within said sheath (1) may inhibit almost all bacteria therearound evidenced by experiment reports provided by such as super laboratory of Yang Ming University incubation center.

Generally, germicidal additives added to products with germicidal effect may be kind of inexpensive material, but safety in germicidal additives are in doubt. While said nano germicidal (7) does not generate toxicity, or cause bare human skin break out in a rash.

Up to the present, germicidal additives added to said elastomer material (11) of said latex (12) with temperature endurance effect is preferably no other choice but nano germicidal (7) of the present invention. Physical attribute may be specified for general nano sized materials to be more activated, but much less temperature endurance so that they are not suitable for adding to said latex (12). While said nano germicidal (7) of the present invention still retains original attribute with sufficient safety to users.

Irradiation of far infrared rays of said nano scale titanium dioxide photocatalysis coating liquid (2) has a wave length in the range of 6-12 μm close to the 9.36 μm, which is released from human beings. The radiation thus can go deep into hypoderm and resonate water molecules in our body then dilate the vein by vibrating water molecules. The water molecules on the surface of cell are active so it will accelerate the flow of calcium ion then advance blood circulation, improve metabolism, active cell tissue, accelerate absorption of oxygen.

Said lubricating germicidal solution (5) is a mixture of said nano scale zinc oxide (3) and said nano scale chitin beads and chitosan beads (4), said nano germicidal (7) is to be directly coupled to cell wall of said bacteria, and invade into the bacteria to inhibit both metabolism and activity of enzyme.

Said elastomer material (11) selected from one of the following: natural rubber, synthetic rubber. Added with said nano scale titanium dioxide photocatalysis coating liquid (2) with far infrared rays, and said nano scale zinc oxide (3) and said nano scale chitin beads and said chitosan beads (4), either natural rubber or synthetic rubber can be nano germicidal-mediated 100% effective on germicidal effect.

What is claimed is:

1. Method of manufacturing a long-acting germicidal condom comprises the steps of:
first step: a nano scale titanium dioxide photocatalysis coating liquid (2) to emit far infrared rays, a nano scale zinc oxide (3) with germicidal effect, and a nano scale chitin beads and chitosan beads (4) are combined to form a composite material (10);
second step: said composite material (10) is mixed into an elastomer material (11) to form a latex (12);
third step: said latex (12) with a density is extruded to form a sheath (1) further coupled with an O ring; and
fourth step: a cylindrical surface of said sheath (1) is spread with lubricating germicidal solution (5); thus to finish a long acting germicidal condom.

2. Method of manufacturing a long-acting germicidal condom of claim 1 wherein said nano scale titanium dioxide photocatalysis coating liquid emits said far infrared rays wave length in the range of 6-12 μm.

3. Method of manufacturing a long-acting germicidal condom of claim 1 wherein said lubricating germicidal solution (5) is a mixture of said nano scale zinc oxide (3) and said nano scale chitin beads and chitosan beads (4).

4. Method of manufacturing a long-acting germicidal condom of claim 1 wherein said elastomer material (11) selected from one of the following: natural rubber, synthetic rubber.

5. A long acting germicidal condom comprises a sheath (1) has a filmy structure to protect male sexual organ, said sheath (1) includes an extruded latex (12) combined with a nano scale titanium oxide photocatalysis coating liquid (2), a nano scale zinc oxide (3) and a nano scale chitin beads and chitosan beads (4);
a lubricating germicidal solution (5) spread on a cylindrical surface about said sheath (1); and
an O ring (6) coupled to an open end of said sheath (1).

6. The long acting germicidal condom of claim 5 wherein said nano scale titanium oxide photocatalysis coating liquid (2) emits far infrared rays wave length in the range of 6-12 μm.

7. The long acting germicidal condom of claim 5 wherein said lubricating germicidal solution (5) is a mixture of said nano scale zinc oxide (3) and said nano scale chitin beads and chitosan beads (4).

8. The long acting germicidal condom of claim 5 wherein said elastomer material is selected from one of the following: natural rubber, synthetic rubber.
